(19) United States
(12) Patent Application Publication
(10) Pub. No.: US 2012/0064619 A1
(43) Pub. Date: Mar. 15, 2012

(54) Device for the Anaerobic Fermentation of Organic Material

(76) Inventors: Luc Albert De Baere, De Pinte (BE); Piet Lodewijk Smis, Gavere (BE); Jan Remi Smis, Merelbeke (BE)

(21) Appl. No.: 13/272,850

(22) Filed: Oct. 13, 2011

Related U.S. Application Data
(63) Continuation-in-part of application No. 11/987,940, filed on Dec. 6, 2007.

(30) Foreign Application Priority Data
Dec. 7, 2006 (BE) 2006/0602

(51) Int. Cl. C12M 1/00

(52) U.S. Cl. 435/290.4

(57) ABSTRACT

A device for implementing anaerobic fermentation having a vertically standing fermentation tank with a fermentation chamber in which organic material can be fermented, a feed device to mix fresh organic material with inoculant and to carry it through to an inlet in the fermentation tank, which is also provided with a cone with an extraction outlet via which fermented material can be discharged and an outlet for biogas, where the device is also provided with two or more return openings via which a fraction of the partly fermented material, located between the inlet and the outlet, is removed from the fermentation tank and is carried up through feed lines between the mixing pump and the inlet that are situated partly and vertically in the fermentation tank.

### CROSS REFERENCE TO RELATED APPLICATION

This application is a continuation-in-part of application Ser. No. 11/987,940, filed on Dec. 6, 2007, which claims priority to Belgian application No. 2006/0602, filed on Dec. 7, 2006, the entirety of which is incorporated by reference.

### FIELD OF THE INVENTION

The present invention concerns a device for the anaerobic fermentation of biodegradable organic material, whereby the fresh organic material is mixed outside the digester with an amount of material which has been partly fermented as an active inoculant for the anaerobic fermentation, and whereby the active inoculant is removed through return openings in a cone, hanging under a vertical digester, before the final extraction outlet, providing an even flow downward through the unmixed dry digester, and creating a post-fermentation in the zone below the side extraction return openings and the final extraction outlet.

The organic material is in this case biodegradable, non-liquid material, in particular various farm crops, either or not specifically cultivated for the production of energy, or the organic fraction of domestic waste, or of similar industrial waste, or other organic fractions, such as for example the sludge of water treatment plants, sludge of the paper industry, green waste, garden waste, organic waste flows from the production of bio-energy from farm crops, or other biologically decomposable fractions comprising at least 15% of dry matter or that can be piled up.

### BACKGROUND

In general, there are different ways of anaerobic fermentation. Thus, said organic materials can be fermented under wet conditions (maximally 5 to 10% dry matter in the fermentation tank) or under dry conditions (more than 15% dry matter in the fermentation tank), under either mesophilic (circa 35 to 45° C.) or thermophilic (circa 48 to 60° C.) conditions.

For the fermentation of organic material under wet conditions, this implies that large amounts of water are added to the organic material in order to obtain a liquid slurry in the fermentation tank of the wet systems, as a result of which the content of the fermentation tanks can be easily mixed internally and the fermented mass can be easily discharged via overflow or simply by pumping.

For the dry systems, the amount of water is restricted to a minimum, or even no water at all is added, so that a pasty mass is obtained. This pasty mass can then be fermented in fermentation tanks that have been especially designed for dry fermentation, with a dry matter content of more than 15%. Since the high viscosity of the fermenting material in a dry fermentation with more than 15% of dry matter does not promote a smooth mixing, special mixing systems are built in fermentation tanks that either provide for a mixing over the entire length of the fermentation tank (several mixers in the different zones or a single mixer over the entire length), or the material to be mixed is mixed outside the fermentation tank in a limited volume. Further, in some of these dry systems, biogas is injected in different zones so as to produce a mixing in these zones.

In wet fermentation systems, fresh water or recycled processing water is either mixed together with the organic material and pumped into the fermentation tanks, or the organic material is pushed directly into the fermentation tank and fresh water, recycled processing water or wet, liquid organic flows are pumped into the fermentation vessel as well, whereby the ingoing mass is mixed over the entire fermentation tank so as to obtain a homogeneous liquid mass. The aim hereby is always to obtain a highly liquid pulp or “slurry” that can be easily pumped and that can be easily mixed in the fermentation tank.

These wet fermentation tanks usually work according to the principle of a completely mixed reactor whereby the ingoing mass is entirely mixed with the fermenting material over the entire volume of the fermentation tank by means of mixing gears or gas injection in the fermentation tank.

Given the liquid pulp or slurry in the fermentation tanks and the intensive internal mixing, the fermenting material and the freshly added material mix fast through the fermentation tank as a whole.

As a result, a part of the freshly added material will possibly be removed from the fermentation tank within a very short time span, along with the fermented digestate. In other words, a piece of fresh food may be removed from the fermentation tank within a time span of only a few minutes, whereas the average residing time in the fermentation tank of the material to be fermented may amount to 20 to 30 days.

In order to solve or at least restrict this problem, as described in patent document WO 98/24730, a pre-chamber was built in that is partly separated from the rest of the completely mixed fermentation tank by a wall. The freshly added material is first fed into this pre-chamber, which preferably has a residing time of several days, before the material ends up in the actual fermentation space. In this way, there is already some fermentation in the pre-chamber with little risk that the supplied material will leave the fermentation tank almost immediately via the outlet together with the rest of the fermented mass as a result of the continuous mixing in the fermentation tank.

Another known solution to the above-mentioned problem is described in EP 0 066 582, which represents a fermentation plant for organic materials operating with a dry matter content of 6% and at a temperature of 55 to 60° C. in several reactors that are driven in an aerobic or anaerobic manner. After the anaerobic fermentation, the fermented material is normally pumped to a storage tank for the residual slurry. In a special embodiment of this device, said storage tank is operated in an anaerobic manner as an after-fermentation tank, such that additional biogas is produced from the fermented material. This requires an entirely separate additional fermentation tank, however.

Apart from the wet fermentation, as mentioned before, also dry fermentation as a method for the anaerobic fermentation of organic waste is known.

In a dry fermentation method, the amount of water that is added is limited, such that there is a relatively solid mixture in the ‘dry’ fermentation vessel which moves through the fermentation tank according to the principle of a plug flow. In order to process organic fractions of domestic waste with high contents of dry matter, for example more than 25% in the fermented digestate, an intensive mixing of the fermenting material is no longer possible in the fermentation vessel, such that the fresh organic material will have to be pre-mixed, externally to the “dry” fermentation vessel, with already fermented material by means of special mixing units. Next, the thick mixture is pumped or pushed into the fermentation vessel by means of special pumps. Other dry fermentation systems operate with contents of dry matter of 15% and up to 25% in the fermented material, which makes it possible to mix in zones by means of different mixers or in zones where gas is injected via the bottom, but only with special mixing systems. The dry fermentation tank can also be designed such that one mixer can mix the total mass with a content of dry matter of less than 25% in the fermented material when processing selectively collected organic fractions from domestic waste. For organic fractions from domestic waste, the content of dry matter of the mixture of organic waste and fermented inoculant that is supplied to the tank is situated between 15 and 50%, more specifically between 20 and 45%. For other organic fractions such as dehydrated slurry, mixing in the fermentation tank is no longer possible as of 15 to 20%, and mixing outside the fermentation tank must already be applied as of 20% of dry matter in the fermented residue. The average content of dry matter in the dry fermentation tanks amounts to more than 15%, whereby the material is drier as it enters the fermentation tank than when it leaves the fermentation tank due to the transformation of dry matter into biogas.

U.S. Pat. No. 6,905,601 describes how this mixture of fresh organic material and fermented material is pumped into the top of a standing fermentation tank by means of feeding tubes. The supplied mixture descends from the top to the bottom during the dry fermentation process, whereby the fermented material is removed from the bottom of the fermentation tank. This process for removing fermented material uses what is called a plug flow, whereby the material that is first supplied to the tank leaves it first as well according to the FIFO principle (First In-First Out).

In this type of reactor fresh material is mixed with fermented material collected at the lowest point of the conical bottom of the fermentation tank, and this mixture is introduced at the top of the feeding tube in the fermentation tank after it is expanded until its density approximates the density of the already present fermenting mass at the top of this downward movement. This expansion happens in the feeding tube, which is a tube, open ended at the top, through which the mixture is pushed upwards and where the mixture is released at the top and inside of the fermentation tank and is pushed into the digester by the action of the feeding pump. Three feeding tubes are provided to allow an even distribution of the feeding in the digester.

A drawback of this type of reactor is that fermented material is taken at the very bottom of the fermentation tank, which is not the zone of most active fermentation but is rather a post-fermentation zone.

Another drawback of this type of reactor is that the extraction at the bottom of the conical tank takes place from a single extraction hole, whereby the extraction may not be optimal for extracting at an even rate. Digestate straight above the extraction hole will move downwards faster than digestate closer to the walls of the cone. This will result in an uneven flow from top to bottom, disturbing the first-in first-out principle.

Another drawback is incomplete digestion resulting in a loss of methane product as a source of renewable energy.

In this type of dry fermentation, a sufficiently large fraction of fermented material must be mixed with the freshly supplied organic material outside the fermentor, for example five units of inoculant per unit of fresh organic material, so as to make sufficient contact between the anaerobic bacteria and the fresh organic material, since mixing is no longer possible after the mixture has been introduced in the dry anaerobic fermentor.

As a major part of the fermented material is recycled, the pass-through time through the fermentation tank is reduced and, depending on the recycled amount, the pass-through time will then amount to some 10 days or even 2 to 3 days. In this way, a part of freshly supplied material will be discharged together with the fermented material after 10 or even two or three days of fermentation, while the average residing time in the fermentation tank amounts to some 12 to 30 days or more. This is already a major improvement compared to the completely mixed wet fermentation tanks, however, since the supplied material is guaranteed to stay 2 to 3 days or even a week in the fermentation tank, as opposed to the almost immediate removal that may occur in these simple liquid systems.

When fermenting fresh organic materials with a high production of biogas per ton, the average residing time in the fermentation tank may increase to 50 to 100 days and more. The pass-through time only amounts to 10% or even 2 to 3% of the average residing time then, as the pass-through time is kept constant.

When the dry fermentors are heavily loaded, such as for example when fermenting high-energy crops such as maize at a high loading rate, the fermented material, which is supposed to have fermented completely as with the known methods for anaerobic fermentation of organic material, still produces a limited amount of biogas. This represents a loss of renewable energy.

Also, when applying such known methods, germs may survive after a short pass-through time in case they are immediately removed from the fermentor and are discharged for subsequent treatment together with the rest of the digestate.

### SUMMARY

The invention is directed to a device for the anaerobic fermentation of organic material which does not have the above-mentioned and other disadvantages.

To this aim, two or more return openings are provided in the conical part of the digester, preferably away from the top of the cone and away from the bottom of the cone, but also in between the feeding tubes so as to create an even downflow from the top of the feeding tubes to the return openings. In addition, the return openings are preferably positioned slightly lower on the cone than the entrance of the feeding tube into the cone, as shown in FIGS. 1. and 2. This allows an evenly distributed extraction from top to bottom of partially fermented material.

To this aim, the anaerobic fermentation of organic material starts with organic material to be fermented which is mixed with inoculant and supplied into a vertical fermentation tank and which moves from an inlet of the fermentation tank to an extraction outlet at the bottom of a cone thereof, whereby the fermented material is removed from the tank via the extraction outlet, wherein a fraction of the fermenting material which is situated between the inlet and the extraction outlet is removed earlier from the fermentation tank via several return openings, at least two but preferably three or more, spread over the surface of the cone and in between the wall and the extraction outlet at the bottom of the cone, and is used as inoculant, while the fermenting material between the side outlet return openings and the bottom extraction outlet, which is the post-fermentation zone, is still post-fermented for a certain time before it is removed from the fermentation tank via the extraction outlet at the bottom of the cone.

Thanks to the application of the device according to the invention, a more evenly distributed downflow is obtained from top to bottom through extraction via the side return openings, improving critical first-in first-out requirements. Also the minimum pass-through time is increased so that a piece of fresh organic material will stay longer in the fermentation tank as the residing time is extended in the post-fermentation phase in case such a piece of organic material is discharged to the after-treatment via the outlet after the first passage; the energy recovery is maximized as the biogas further produced during the post-fermentation is collected.

The partly fermented material is further fermented between the return opening and the extraction outlet in the lower part of the cone for an additional length of time until a stable, fermented digestate is obtained within one and the same reactor. Thanks to this internal post-fermentation where the digestate is allowed to ferment without the addition of fresh feedstock, which takes place in the post-fermentation zone in the fermentation tank itself, it is not necessary to provide an additional fermentation tank with accessories, which represents a cost saving. The evenly distributed downflow, together with an additional post-fermentation also provides more certainty as to the killing of germs or weeds, since all germs or weeds that might be present have a longer residing time than if they would be immediately discharged to the after-treatment via the outlet as well.

By providing return openings for the recycling of the inoculant, two zones are created in the fermentation tank, namely a first “active fermentation” zone upstream of the place where a fraction of the partly fermented material is removed from the fermentation tank via the return openings, and a second post-fermentation zone downstream of the latter, whereby the material is removed from the second zone via the outlet for fermented digestate.

The entirely fermented digestate extracted at the bottom of the cone is usually not recycled, but possibly subjected to an after-treatment to produce compost or similar dry end product or taken directly to the fields. If, however, the fermentation would appear to be biologically less stable, an amount of fermented stable material may be added to the inoculant, i.e. the partly fermented material, so as to adjust the biological process.

In the first zone, the organically inoculated material is supplied and an aerobically fermented. A part of this fermenting material which is situated in the first fermentation zone is recycled via the return openings in the side of the cone and mixed as an inoculant with fresh organic material to be fermented. The rest of the fermenting material ends up in the second zone downstream of the return opening, namely the post-fermentation zone.

Partly fermented material which is situated in the second zone has passed the first fermentation zone, maybe several times, as it has been recycled one or several times and has been used as inoculant. As soon as it ends up in the second zone, the partly fermented material is degraded for an additional two to four days and does no longer become optimal for recycling as an active inoculum. It steadily moves further to the extraction outlet of the fermentation tank, preferably in a plug flow, depending on whether entirely fermented material is removed from the fermentation tank and becomes less and less active.

The partly fermented material is hereby subjected to an after-fermentation that occurs in this second phase in the same fermentation tank and that comes down to a mere finishing of the methanogenic phase during a period in which no additional material is being fed. The biological activity quickly decreases as the material approaches the extraction outlet. The material stays with certainty for a certain minimum time in this second zone, and in the end it is discharged via the outlet.

Preferably, the volume of the second post-fermentation zone amounts to at least one fiftieth of the total volume of the fermentation tank, such that there is sufficient volume in the second zone for an after-fermentation of at least half a day to even a few days, for example 2 to 4 days or more if useful.

An additional advantage of applying such a device according to the invention is that the partly fermented material which is removed from the tank via the return openings on the side of the cone and which is afterwards recycled and used as an inoculant, is even more biologically active than the entirely fermented material which is used as an inoculant in the known devices e.g. in U.S. Pat. No. 6,905,601.

Also the characteristics of the fermented material will have changed more than those of the partly fermented material following the after-fermentation. For example, the partly fermented material which is removed via the return openings as an active inoculant has a pH of 7.5 to 7.8, whereas the entirely fermented material has a pH of 8.2 to 8.5. During the feeding, the pH drops to about 7, such that the acidity transition, what is called the pH shock, is less large with the partly fermented material than would be the case if the entirely fermented material were to recirculate before being mixed with the fresh organic material.

By selecting the right place for draining the inoculant via the return opening, a maximally active inoculant can be recycled, and by providing a sufficiently large volume for the after-fermentation for the partly fermented material which is not recycled as an inoculant, downstream of the return opening, it is possible to produce an optimally stabilized fermented material. Further, an optimal amount of biogas can be recycled during the after-fermentation in the two phases.

If the fermentation would begin to function less optimally from a biological point of view, part of the entirely fermented material which is discharged via the extraction outlet at the bottom of the cone could still be added to the mixture of inoculant and fresh organic material to be supplied so as to obtain an additional inoculation. In this way, the fermentation can be quickly adjusted by partly limiting the after-fermentation. The volume in the second post-fermentation zone may then be regarded as a reserve of intensive fermentation capacity. If necessary, it is possible to use only entirely fermented material as an inoculant with the fresh organic material to be supplied, for example so as to compensate for seasonal fluctuations or biological imbalances.

The present invention concerns a device for implementing said fermentation method, and to that end it comprises a vertically standing fermentation tank in which organic material can be fermented, a supply device which can mix fresh organic material with inoculant and can pump and feed it through inlet feeding tubes in the fermentation tank, which is also provided with an extraction outlet at the bottom of the cone underneath the fermentation tank, via which fermented material can be discharged, as well as with an outlet for biogas, and whereby the device is also provided with return openings via which a fraction of the fermenting material, situated between the inlet and the extraction outlet, can be removed from the fermentation tank and transported to the feed device, and which return openings are evenly distributed over the cone so as to insure an even downflow through the tank.

### BRIEF DESCRIPTION OF THE DRAWINGS

In order to better explain the characteristics of the invention, the following preferred embodiment of a device for the anaerobic fermentation of organic material according to the invention is described as an example only without being limitative in any way, with reference to the accompanying drawings, in which:

FIG. 1 schematically represents an embodiment of a device for the anaerobic fermentation according to the invention, seen as a section;

FIG. 2 is a section according to line II-II in FIG. 1.

### DETAILED DESCRIPTION OF VARIOUS EMBODIMENTS OF THE DISCLOSURE

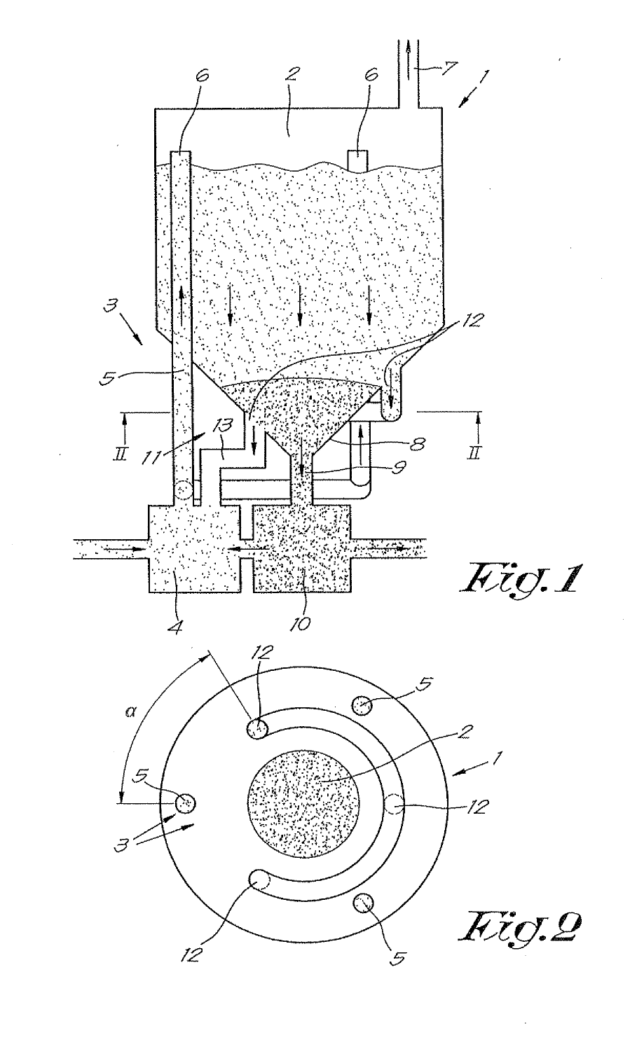

The device for the anaerobic fermentation of organic material, represented in FIG. 1, mainly consists of a vertically standing, closed fermentation tank 1 comprising a fermentation room 2 and a feed device 3 which in this case consists of a mixing pump 4 for mixing fresh organic material with inoculant and for pumping this mixed mass, which in this case opens via feed lines 5 in an inlet 6 inside near the top of the fermentation tank 1. At the top, the fermentation tank 1 is also provided with an outlet 7 for biogas.

At the bottom of the vertical fermentation tank 1 is a conical part and, in the center of the bottom 8 of the cone, an extraction outlet 9 is provided.

This extraction outlet 9 opens in an extraction pump 10 which can discharge the entirely fermented material, or recycle the fermented material towards the mixing pump 4.

According to the invention, the fermentation tank 1 is provided with return openings 12, at least two but preferably three or more, between the top of the cone and the outlet 9 which make a more even extraction of the contents of the fermentation tank possible and make it possible to discharge a fraction of the partly fermented material, situated between the inlet 6 and the extraction outlet 9, from the fermentation tank 1 and carry it via recycling lines 13 to the feed device 3, in this case to the mixing pump 4, where it is mixed with freshly supplied organic material before the thus obtained mixture is put in the fermentation tank 1 via the inlet 6.

In this embodiment, the return openings 12 are situated at a height above the outlet 9 in the conical part of the tank 1, in particular such that the volume occupies one fifth of the total volume of the fermentation tank 1 downstream of the return opening 12.

Return openings 12 that are adjustable in height can possibly be provided, such that the return openings can be positioned at different locations on the side of the cone, and such that partly fermented material can be recycled after varying residing times.

The working of such a device, as discussed above and as represented in FIG. 1, as well as the method for fermenting organic material in an anaerobic manner is simple and is illustrated below by means of an example in FIG. 1.

### EXAMPLE

Starting with a vertical fermentation tank 1 having a total fermentation room of 2000 m³ (active volume including the cone) that is filled with fermenting organic material, 100 m³ of fresh organic material is mixed with 300 m³ of partly fermented material which is used as an inoculant during the daily feeding. It is assumed that the amount of water and/or steam supply required to obtain the desired content of dry matter in the mixing pump is equal to the tonnage of wet biogas that is produced.

In this way, 300 m³ of partly fermented material is removed daily from the fermentation tank 1 via one of the three return openings 12 in the cone and the recycling line 13. Since the recycling line 13 opens at a height above the extraction outlet 9 in the tank 1, the inoculant is formed of partly fermented material which is rich of active bacteria, as opposed to the organic material which is entirely decomposed when it is extracted from the fermentation tank 1 via the extraction outlet 9, whereby the bacteria have already become significantly less active. The pass-through time with recycling via return openings 12 is four days, instead of five days if there were no recycling via these return openings 12.

The fresh material and the inoculant are pushed further and are mixed by action of the mixing pump 4.

Via the feed lines 5, the mixture is fed into the fermentation tank 1 via the inlet 6.

Thanks to an appropriate mixing ratio of the fresh organic material and the inoculant, the mixture is sufficiently pumpable and it can be pumped or carried into a closed fermentation tank. Also, the mixture is amply provided with anaerobic bacteria, such that the fermentation process can start immediately and without any notable delay.

Indeed, thanks to the fact that the inoculant is formed of partly fermented organic material which is removed from the fermentation tank at a distance above the extraction outlet 9, the matter is bacterially more active.

In the fermentation tank 1, the mixture is added to the fermenting mass and moves in the direction of the outlet 9. Partly fermented material that is possibly further recycled via the recycling lines 13 to be used as an inoculant is situated in the zone upstream of or above the recycling lines 13, which hereafter is called the first zone. This first zone in this example represents a volume of some 1600 m³.

Since an average of 400 m³ of mixed material per day is pumped in at the top of the fermentation tank and the first zone, and also 300 m³ of partly fermented material is removed from the first fermentation zone via the recycling lines 13, and 100 m³ of entirely fermented material is removed from the post-fermentation zone 2 at the bottom of the cone of the fermentation tank 1 via extraction outlet 9 as well, so that 100 m³ of partly fermented material simultaneously drops through from the first fermentation zone to the post-fermentation zone, a residing time of four days is obtained. After this average residing time of four days, the provided mixture of organic material and inoculant is removed from the fermentation tank 1 via the recycling lines 13 as partly fermented material which will be added in the mixing pump 4 as inoculant.

Partly fermented material which is not removed from the fermentation tank 1 at the recycling line 13 moves further down in the direction of the outlet 9. In this post-fermentation zone, which represents a volume of some 400 m³, the partly fermented material is no longer optimal to be recycled. It slowly drops further in a plug flow to the outlet 9 at the bottom of the cone of the fermentation tank 1. The material is hereby subjected to an after-fermentation that is carried out in the same fermentation tank 1 and simply comes down to the methanogenic phase being finished, whereby no additional material is being fed.

The biological activity decreases as the material approaches the extraction outlet 9. The material in this case resides for another 4 days in this second zone B, since 100 m³ of entirely fermented material must be daily removed via outlet 9 to make room for the 100 m³ of fresh organic material that is daily added to the fermentation tank 1.

The extraction pump 10 removes the fermented material for after-treatment.

The average residing time amounts to twenty days in this embodiment, since 100 m³ of fresh organic material is fed to the plant, but with an internal recycling time of the partly fermented material of four days in the first fermentation zone and an after-fermentation of four days in the post-fermentation zone, this amounts to a minimum residing time of eight days for any piece of organic material that is fed to the fermentation tank.

On average, the partly fermented material is recycled four times as an inoculant. It is possible, however, that a piece of freshly fed organic material accidentally passes the outlet as of the first time and is not recycled. This piece of organic material will then nevertheless be post-fermented for another 4 days, which amounts to a minimum residing time of eight days. Other pieces of organic material will be recycled two to six times and more so as to be inoculated. If no division in two zones and additional phases had been provided, a recycling time or pass-through time of five days would have been obtained with this fermentation tank 1.

By providing return openings 12 and a recycling line 13, a volume of 400 m³ is created for the post-fermentation, which has for a result that the minimal guaranteed residing time is raised from five to eight days, without a second separate fermentation tank being required.

The biogas that is produced in the fermentation tank 1 is discharged via the outlet 7 for biogas that is provided at the top of the fermentation tank 1.

It is clear that the mixing pump 4 can be replaced by a mixer and a pump, or any system whatsoever to partly mix the material, and a system or device to carry the mixed material to the inlet 6 of the fermentation tank 1, or a system whereby the fresh organic material and partly fermented material are put together or are supplied to the fermentation tank 1 via a separate inlet in a specific proportion, even without any active mixing. It is also possible to install a mixer or several mixers (mechanical or with gas) in the active first fermentation zone and in the ultimate post-fermentation zone, but in such a manner that both zones cannot be mixed, i.e. that material situated after the recycling line 13 is not mixed again with material situated in front of the recycling line 13.

It is also clear that, if in a fermentation tank 1 having the same volume of 2000 m³, the recycling lines 13 were positioned closer to the outlet 9, for example such that the first fermentation zone occupies a volume of 1700 m³, whereas the post-fermentation zone then occupies a volume of 300 m³, and with a supply and a corresponding discharge of 150 m³ and a recycling volume of 700 m³, a recycling time of two days would be obtained, a post-fermentation time of two days and a total minimal pass-through time of four days, whereas the average total residing time would then amount to 2000:150=13.33 days. In this way, it is possible to maintain a very high load, while nevertheless fermented material is being produced that has resided in the fermentation tank for minimally four days, instead of 2000:850=less than two and a half days.

It is obvious that the feed device 3 may comprise means which determine the proportion of the fresh organic material to the recycled inoculant, and that this proportion can be set or adjusted by means of a control.

It is also clear that the volumes of the zones, the recycling ratios and the average residing time should be adjusted and optimized depending on the organic material to be processed, the desired organic load, and the desired production of biogas as well as the stability of the partly or entirely fermented material.

Finally, it is also clear that a fermentation tank 1 according to the invention can also be provided with return openings 12 at various distances between the inlet 6 and the outlet 9, and with accompanying recycling lines 13 going to the feed device 3, whereby an additional zone is created per additional return opening 12 between the above-mentioned first fermentation and post-fermentation zones. The material in every zone has specific characteristics that can provide for a desired effect by an appropriate control of the accompanying return of organic material.

FIGS. 1 and 2 represent a device for the anaerobic fermentation of organic material according to the invention, wherein the feed lines 5 between the mixing pump 4 and the inlet 6 are situated partly and vertically in the fermentation tank 1. The feed lines 5 as well as the recycling lines can be partly horizontal, depending on the position of the mixing pump 4.

The return openings 12 consist of several openings 12 that lead to the mixing pump 4 via recycling lines 13.

Also these recycling lines 13 between the mixing pump 4 and the connection to the bottom 8 of the fermentation tank 1 run vertically, in particular up to a height above the extraction outlet 9 in the conical part of the fermentation tank 1.

FIG. 2 illustrates how three feed lines 5 are provided, here each at a mutual angular displacement of 120° , and how three recycling lines 13 are provided in a similar manner, each time one at an angular displacement a, in this case at 60° in relation to a feed line 5.

The feed lines 5 are situated near the standing wall of the fermentation tank 1 in this embodiment, whereas the recycling lines 13 are situated somewhat closer to the central outlet 9.

This can also be reversed, whereby the recycling lines 13 are built in closer to the wall, and the feed lines 5 more centrally in the conical part of the fermentation tank 1.

The feeding may possibly be provided for via one or several points in the roof or at the top of the reactor via external feed lines. Further, the recycling lines can be provided higher or lower in the conical part.

Thanks to the special mutual positioning of the above-mentioned lines, well distributed and in this case at a mutual angular displacement of 60° , whereby the return openings and recycling lines are situated somewhat more centrally and slightly lower on the cone than the feeding tubes, a good flow-through of the fermenting material and a good even distribution of recycled material at the top of the fermentation tank as well as an even extraction at the bottom of the fermentation tank is obtained.

As represented in FIGS. 1 and 2, the return openings 12 may be provided at mutually different distances from, or in this case at different heights above the extraction outlet 9. Possibly, the distance of return opening 12 to the outlet 9 is adjustable.

It is clear that for the discussed embodiment, the feed device 3 can also be built differently, and may contain for example a separate pump and mixer, or the pump may be replaced by other means to propel the fresh organic material and the inoculant in a certain proportion, possibly without any active mixer.

The fresh organic material can also be added to the fermentation tank 1 or to the feed line 5 via a separate pump, just as the partly fermented material can be added separately via another pump and feed line 5. A mixer or pusher screw can be additionally built in, in the fermentation tank 1 to either mix or propel the material. Biogas can also be injected to partly propel and/or mix the fermenting material. Preferably, the horizontal push or mixing system is conceived such that there is no or only a limited mixing between the first fermentation zone and the second post-fermentation zone.

It is also clear that the return opening 12 can be connected to the feed device 3 in different ways, and that the recycling lines 13 can be replaced by other means that can provide for the transport of partly fermented material as an inoculant.

The invention is by no means restricted to the embodiments described above and represented in the accompanying drawings; on the contrary, such a device for the anaerobic fermentation of biodegradable material can be made in all sorts of variants while still remaining within the scope of the invention.

1. A device for implementing anaerobic fermentation comprising:
a vertically standing fermentation tank having a fermentation chamber, an inlet, an outlet, and a cone, wherein said fermentation chamber is configured to ferment organic material;
a feed device configured to mix fresh organic material with inoculant to form a mixture and configured to carry said mixture to the inlet of the fermentation tank,
wherein said cone has an extraction outlet configured to discharge fermented material and an outlet configured to discharge biogas,
wherein the device further comprises two or more return openings configured to remove from the fermentation tank a fraction of the partly fermented material located between the inlet and the outlet of the cone, and configured to carry up said fraction through feed lines between a mixing pump and the inlet of the fermentation tank, where said feed lines are situated partly and vertically in the fermentation tank.

2. The device according to claim 1, wherein the feed device comprises a pump.

3. The device according to claim 1, wherein the feed device comprises a control that determines the proportion of fresh organic material to recycled inoculant.

4. The device according to claim 1, wherein the fermentation tank comprises a conical bottom with at least two or more return openings and recycling lines, each one of said two or more return openings and recycling lines have a same angular displacement in relation to the feeding lines and at a position slightly lower than an entrance of the feeding lines into the cone of the fermentation tank.

5. The device according to claim 1, wherein the fermentation tank comprises return openings that are adjustable in height, such that the return openings can be positioned at different locations on the side of the cone, and such that partly fermented material can be recycled after varying residing times.

6. The device according to claim 1, wherein recycling lines are provided between the return opening and the feed device.

7. The device according to claim 1, wherein a return line is provided between the extraction pump and the feed device.
